(19) United States
(12) Patent Application Publication
(10) Pub. No.: US 2026/0060558 A1
(43) Pub. Date: Mar. 5, 2026

(54) ADAPTERS, CONNECTORS, AND TUBING FOR PRESSURE CUFF

(71) Applicant: Medline Industries, LP, Northfield, IL (US)

(72) Inventors: Zachary Rzeszutek, Chicago, IL (US); Lauren Pritz, Champaign, IL (US); Steven V. Bisbikis, Northbrook, IL (US)

(21) Appl. No.: 18/819,939

(22) Filed: Aug. 29, 2024

(51) Int. Cl. A61B 5/022; A61B 5/021

(52) U.S. Cl. CPC A61B 5/02233; A61B 5/02141

(57) ABSTRACT

Adapters, connectors, and tubing provide for connection of a blood pressure cuff to a pressure source, such as a patient cardiac monitor. Adapters, connectors, and tubing are configured to allow a patient to be connected and disconnected from monitoring equipment without removing the blood pressure cuff from the patient's appendage. Among other aspects, adapters may include various configurations that allow connection of: single lumen tubes to single lumen tubes or equipment; single lumen tubes to dual lumen tubes or equipment; dual lumen tubes to dual lumen tubes or equipment; and dual lumen tubes to single lumen tubes or equipment. Tubes may include dual lumen tubing where the lumens are separated by a web, and the web maintains the spacing between the connectors attached to the lumens.

### FIELD OF THE INVENTION

The present invention relates generally to connectors and tubing for use in medical applications, and particularly to connectors and tubing used in providing air lines for sphygmomanometers or blood pressure monitors.

### BACKGROUND

Blood pressure monitors, also known as sphygmomanometers, are used as non-invasive patient evaluation or monitoring devices. For example, blood pressure monitors may function by inflating a cuff secured around an appendage such as an arm or leg. The cuff occludes arterial blood flow in the appendage around which the cuff is wrapped. Once above systole, the cuff can be deflated, and the systolic and diastolic pressures of the patient can be calculated based on measurements made during cuff deflation. Other indicators or measurements related cardiovascular health may also be observed or monitored by the cuff or ancillary devices, including heart rate, temperature, blood oxygen level (SpO2), PR interval, and electrocardiogram (ECG), among others.

Inflation and deflation of the cuff may be manually controlled by a health care provider using an inflation bulb to pressurize the cuff with a release valve to deflate the cuff. The blood pressure may be observed on a gauge. The various external components, such as the inflation bulb, release valve, and gauge, are fluidly connected to the cuff using one or more sections of tubing that convey air pressure between the cuff and the various components. Alternatively, the cuff may be controlled by, and blood pressure and other indicators may be periodically measured by, an electronic patient cardiac monitor. As with a manual sphygmomanometer, the cardiac monitor is connected to a cuff with one or more air hoses.

Blood pressure is typically measured by wrapping the cuff around the patient's arm or leg and administering a blood pressure test. However, at times, it may be difficult, cumbersome, or time consuming to apply and remove the cuff if blood pressure may need to be measured multiple times. Accordingly, at times it may be advantageous to connect and disconnect a patient from monitoring equipment while leaving the cuff in position on the patient's limb. For example, if a patient is to be monitored for an extended time, it may be advantageous to provide a relatively easier method of connecting and disconnecting the tubing of the cuff to allow patient mobility, or it may be advantageous to allow connection of a single cuff to different monitoring equipment as the patient is moved between various locations. In addition, it may be advantageous to connect the cuff with various monitoring equipment using different connector configurations.

Accordingly, a need exists for cuff, tubing, connectors, and adapters that enable removable connection of air tubes associated with blood pressure cuffs in a manner that overcomes or improves upon known cuffs and connection devices.

### BRIEF DESCRIPTION OF THE DRAWINGS

The foregoing and other advantages of the invention will become apparent upon reading the following detailed description and upon reference to the drawings.

FIG. 1 shows an illustrative embodiment of a manual sphygmomanometer.

FIG. 2 shows an illustrative embodiment of an electronic blood pressure monitor system.

FIG. 3 shows an illustrative embodiment of a disposable blood pressure cuff.

FIG. 4 shows an illustrative embodiment of tubing for use in embodiments of a blood pressure cuff.

FIG. 5 shows a cross-sectional view of tubing consistent with the tubing embodiment illustrated in FIG. 4.

FIG. 6 shows an illustrative embodiment of a connector.

FIG. 7 shows a perspective view of an embodiment of an adapter having a single barbed connector and a single aperture for inserting a connector.

FIG. 8 shows a separate perspective view of an adapter consistent with embodiments of the adapter shown in FIG. 7.

FIG. 9. shows cross-sectional view of an adapter consistent with embodiments of the adapter shown in FIG. 7.

FIG. 10 shows a perspective view of a top case for use with embodiments of the adapter shown in FIG. 7.

FIG. 11 shows a perspective view of a bottom case for use with embodiments of the adapter shown in FIG. 7.

FIG. 12 shows a perspective view of a cap for use with embodiments of the adapter shown in FIG. 7.

FIG. 13 shows a perspective view of an ejector for use with embodiments of the adapter shown in FIG. 7.

FIG. 14 shows a perspective view of a retainer for use with embodiments of the adapter shown in FIG. 7.

FIG. 15 shows a perspective view of a button for use with embodiments of the adapter shown in FIG. 7.

FIG. 16 shows a cross-sectional view of an adapter before a connector has been inserted.

FIG. 17 shows a cross-sectional view of an adapter consistent with embodiments of the adapter shown in FIG. 16 after a connector has been fully inserted.

FIG. 18 shows a perspective view of an embodiment of an adapter having two barbed connectors and a single aperture for inserting a connector.

FIG. 19 shows a separate perspective view of an adapter consistent with embodiments of the adapter shown in FIG. 18.

FIG. 20. shows cross-sectional view of an adapter consistent with embodiments of the adapter shown in FIG. 18.

FIG. 21 shows a perspective view of a top case for use with embodiments of the adapter shown in FIG. 18.

FIG. 22 shows a perspective view of an embodiment of an adapter having two barbed connectors and two apertures for inserting connectors.

FIG. 23 shows a separate perspective view of an adapter consistent with embodiments of the adapter shown in FIG. 22.

FIG. 24. shows cross-sectional view of an adapter consistent with embodiments of the adapter shown in FIG. 22.

FIG. 25 shows a perspective view of a top case for use with embodiments of the adapter shown in FIG. 22.

FIG. 26 shows a second perspective view of a top case for use with embodiments of the adapter shown in FIG. 22.

FIG. 27 shows a perspective view of a bottom case for use with embodiments of the adapter shown in FIG. 22.

FIG. 28 shows a second perspective view of a cap for use with embodiments of the adapter shown in FIG. 22.

FIG. 29 shows a second perspective view of a button for use with embodiments of the adapter shown in FIG. 22.

FIG. 30 shows a perspective of a top case for use with embodiments of an adapter having a single barbed connector and two apertures for inserting connectors.

FIG. 31. shows a second perspective view of the top case of FIG. 30.

FIG. 32 shows a cross-sectional view of the top case of FIG. 30.

FIG. 33 shows a perspective view of embodiments of a connector.

FIG. 34 shows a second perspective view of the connector of FIG. 33.

FIG. 35 shows a cross-sectional view of the connector of FIG. 33.

FIG. 36 shows a front view of a connector consistent with embodiments of the adapter shown in FIG. 33 with an adapter attached.

FIG. 37 shows a second perspective view of the connector of FIG. 36.

FIG. 38 is a perspective view of a top side of an embodiment of an adapter.

FIG. 39 is a perspective view of a bottom side of the adapter of FIG. 38.

FIG. 40 is an elevation view of the front side of the adapter of FIG. 38.

FIG. 41 is an elevation view of the back side of the adapter of FIG. 38.

FIG. 42 is an elevation view of the left side of the adapter of FIG. 38.

FIG. 43 is an elevation view of the right side of the adapter of FIG. 38.

FIG. 44 is a plan view of the top side of the adapter of FIG. 38.

FIG. 45 is a plan view of the bottom side of the adapter of FIG. 38.

FIG. 46 is a perspective view of a top side of an embodiment of an adapter.

FIG. 47 is a perspective view of a bottom side of the adapter of FIG. 46.

FIG. 48 is an elevation view of the front side of the adapter of FIG. 46.

FIG. 49 is an elevation view of the back side of the adapter of FIG. 46.

FIG. 50 is an elevation view of the left side of the adapter of FIG. 46.

FIG. 51 is an elevation view of the right side of the adapter of FIG. 46.

FIG. 52 is a plan view of the top side of the adapter of FIG. 46.

FIG. 53 is a plan view of the bottom side of the adapter of FIG. 46.

FIG. 54 is a perspective view of a top side of an embodiment of an adapter.

FIG. 55 is a perspective view of a bottom side of the adapter of FIG. 54.

FIG. 56 is an elevation view of the front side of the adapter of FIG. 54.

FIG. 57 is an elevation view of the back side of the adapter of FIG. 54.

FIG. 58 is an elevation view of the left side of the adapter of FIG. 54.

FIG. 59 is an elevation view of the right side of the adapter of FIG. 54.

FIG. 60 is a plan view of the top side of the adapter of FIG. 54.

FIG. 61 is a plan view of the bottom side of the adapter of FIG. 54.

FIG. 62 is a perspective view of a top side of an embodiment of an adapter.

FIG. 63 is a perspective view of a bottom side of the adapter of FIG. 62.

FIG. 64 is an elevation view of the front side of the adapter of FIG. 62.

FIG. 65 is an elevation view of the back side of the adapter of FIG. 62.

FIG. 66 is an elevation view of the left side of the adapter of FIG. 62.

FIG. 67 is an elevation view of the right side of the adapter of FIG. 62.

FIG. 68 is a plan view of the top side of the adapter of FIG. 62.

FIG. 69 is a plan view of the bottom side of the adapter of FIG. 62.

FIG. 70 is a perspective view of a top side of an embodiment of a connector.

FIG. 71 is a perspective view of a bottom side of the connector of FIG. 70.

FIG. 72 is an elevation view of the front side of the connector of FIG. 70.

FIG. 73 is an elevation view of the back side of the connector of FIG. 70.

FIG. 74 is an elevation view of the right side of the connector of FIG. 70.

FIG. 75 is an elevation view of the left side of the connector of FIG. 70.

FIG. 76 is a plan view of the top side of the connector of FIG. 70.

FIG. 77 is a plan view of the bottom side of the connector of FIG. 70.

The drawings listed above are intended to convey to one of ordinary skill in the art the present invention and its embodiments. In some drawings certain elements have not been shown for clarity. While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

Embodiments of the invention are now described in detail. The following description of various embodiments, as represented in the figures, is not intended to limit the scope of the present disclosure but is merely representative of various embodiments. Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Referring to the drawings, like numbers generally indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, proximal and distal, posterior and anterior, and the like may be used solely to distinguish one entity or action from another entity or action without necessarily requiring or implying any actual such relationship or order between such entities or actions.

Additionally, instances in this specification where one element is “coupled to another element” can include direct and indirect coupling. Direct coupling can be defined as one element coupled to and in some contact with another element. Indirect coupling can be defined as coupling between two elements not in direct contact with each other but having one or more additional elements between the coupled elements. Further, as used herein, securing one element to another element can include direct securing and indirect securing. Additionally, as used herein, “adjacent” does not necessarily denote contact. For example, one element can be adjacent to another element without being in contact with that element. In addition, the term “clinician” is used herein to indicate the medical practitioner or any other person that may utilize a speculum. The term is not intended to be limiting on the scope of the described or claimed embodiments.

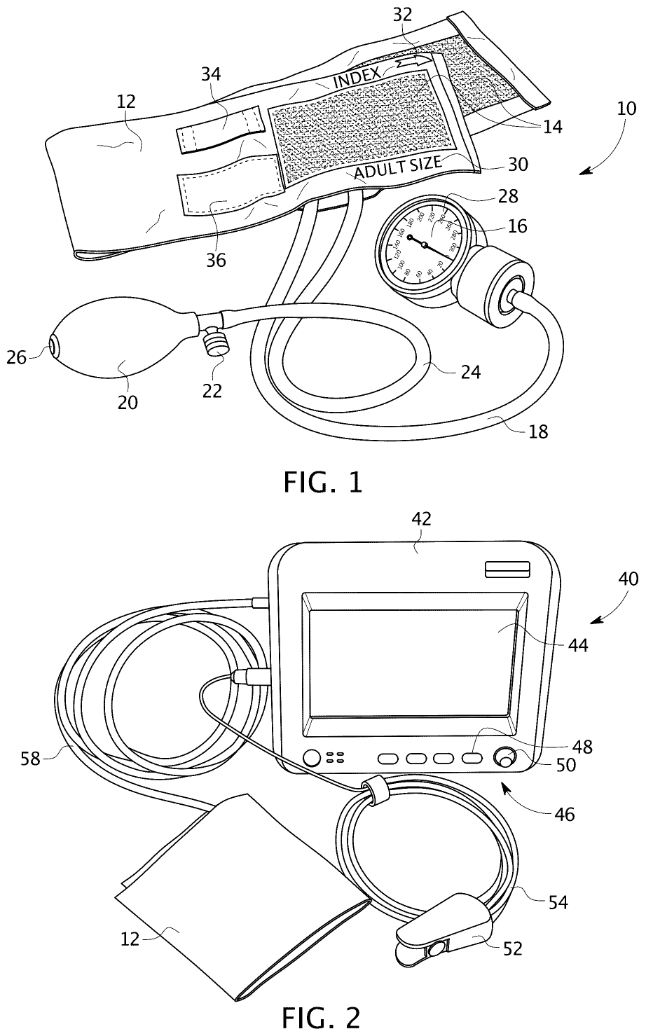

FIG. 1 illustrates an embodiment of a sphygmomanometer or blood pressure monitor 10 that is manually actuated. Embodiments of the sphygmomanometer comprise a cuff 12. The cuff may be a reusable or disposable cuff. The cuff may be adapted to encircle a patient's appendage, such as an arm or leg. The cuff may comprise complimentary hook and loop 14 fasteners or other closure or securement mechanisms. The sphygmomanometer may further comprise a gauge 16 that is fluidly connected with one or bladders disposed in the cuff by a gauge tube 18. The tube may be flexible and may comprise rubber, plastic, thermoplastic elastomer (“TPE”), silicone, or other appropriate materials. The sphygmomanometer 10 may further comprise an inflation bulb 20 that provides inflation air to the cuff bladder. The sphygmomanometer may also comprise an air release valve 22 in fluid communication with the bulb 20 and the cuff. The bulb 20, cuff 12, and release valve 22 may be fluidly connected by an inflation tube 24. The inflation bulb 20 may comprise an end valve 26. The sphygmomanometer 10 may comprise additional elements of features the aid in using the sphygmomanometer and in measuring or monitoring a patient's blood pressure, including appropriate indicia 28 on the gauge 16. The cuff 12 or other elements of the sphygmomanometer may comprise sizing indicia 30 indicating a size or range of sizes for the cuff. The cuff may also comprise indexing indicia 32 to aid in proper placement of the cuff 12. The cuff may also comprise a gauge holder 34 to secure the gauge 16 and an arterial indicator label 36.

Blood pressure monitoring may also be aided by an electronic or computer controlled cardiovascular monitor or by a monitor that automates or otherwise electronically assists measurement and monitoring of blood pressure and other cardiovascular health indicators. Such devices may be referred to herein as blood pressure monitors, but it will be understood by the person of ordinary skill in the art that such blood pressure monitors may comprise additional functions and measure or monitor additional indicators beside blood pressure. Such blood pressure monitors may vary significantly in features and complexity and may provide for electronic control of blood pressure measuring and/or monitoring in various ways based on, among other things, user input, a preprogrammed schedule, alarms or other triggering events, etc. Such monitors need not control all aspects or steps necessary to measure and monitor blood pressure.

FIG. 2 illustrates an embodiment of an electronic blood pressure monitor system 40. The system may comprise a monitor 42 that comprises a display 44 and a user interface 46 that may include buttons 48 and/or dials 50 or other interface elements. The monitor 42 may be connected to a cuff 12 by a tube 58. The tube may have one, two, or more lumens and may connect the cuff 12 with various elements of the monitor, including pumps, sensors, etc. The monitor 42 may comprise a pump or other air pressure generating mechanism to provide pressure to a bladder in the cuff. The system 40 may also comprise additional monitoring elements, including, as illustrated, a pulse oximeter 52 connected to the monitor 42 by a cable 54. The cuff 12 may include features of the cuff discussed with regard to the manual sphygmomanometer of FIG. 1.

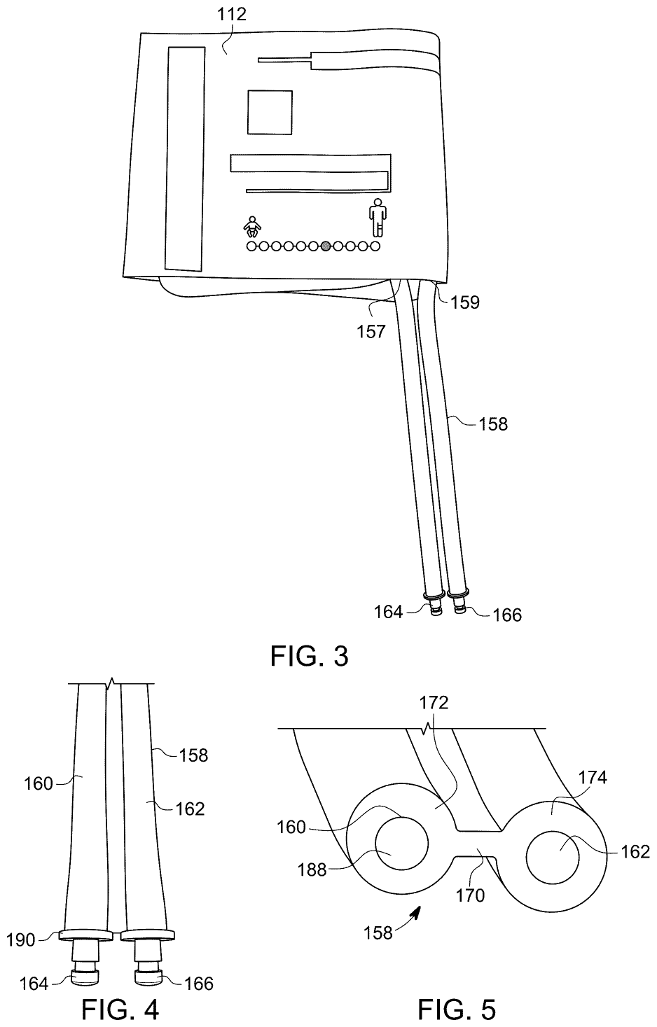

FIGS. 3-5 illustrate embodiments of a blood pressure cuff 112. The cuff may be a disposable cuff and/or may have features discussed above with respect to cuff (12). The cuff may comprise a first air passageway 157 that may function as an inlet or outlet and a second air passageway 159 that may function as an inlet or outlet. The cuff may comprise a tube 158 extending from the cuff and attaching to the air passageways 157, 159. The tube 158 may comprise multiple air channels or lumens, including embodiments comprising a first lumen 160 and a second lumen 162. Each lumen may be generally circular, comprising a central axis. A dual air channel tube embodiment as illustrated in FIGS. 3-5 may facilitate faster blood pressure reading relative to a single channel tube. Connectors 164, 166 may be attached to ends 168 of the tube opposite the cuff 112.

The connectors may connect the tube with an adapter connected to a monitor, as illustrated in FIG. 2. The adapter may be directly connected to or integrated into the monitor or may be connected to a tube extending from the monitor, as will be discussed in greater detail below. The use of connectors 164, 166 with a disconnectable adapter may provide an advantage of allowing the cuff 112 to be separated from a first monitor and later connected to a second monitor without removing the cuff from the patient.

The tube 158 may comprise a first lumen 160 and a second lumen 162 separated by a web 170 that extends between a sidewall 172 of the first lumen 160 and a sidewall 174 of the second lumen. The first and second lumens may have the same or different diameters and may have the same or different sidewall thicknesses. The web may have a consistent length such that a relatively consistent spacing is maintained between the first and second lumens, which in turn may result in a consistent space between connectors 164, 166 attached to the lumens 160, 162. The web may have a thickness that is sufficient to maintain the consistent spacing between the lumens. In some embodiments, the web thickness may be less than a wall thickness of lumen sidewalls 172,174. Alternatively, the web thickness may be greater than the wall thickness but less than an outside diameter of one or both lumens. Alternatively, the web thickness may be equal to the outside diameter of one or both lumens.

In this manner, a dual lumen tube, such as the tube illustrated in FIGS. 3-5 may have an advantage of being easily connected with a dual port connector having the same consistent spacing when compared with two separate, single lumen tubes. The connectors 164, 166 may be directly connected to each other or may be not directly connected to each other such that the spacing between the connectors is controlled by the spacing of the lumens as determined by the length of web 170. The tube, including lumens 160, 162 and web 170 may be extruded as a single, integrated extrusion.

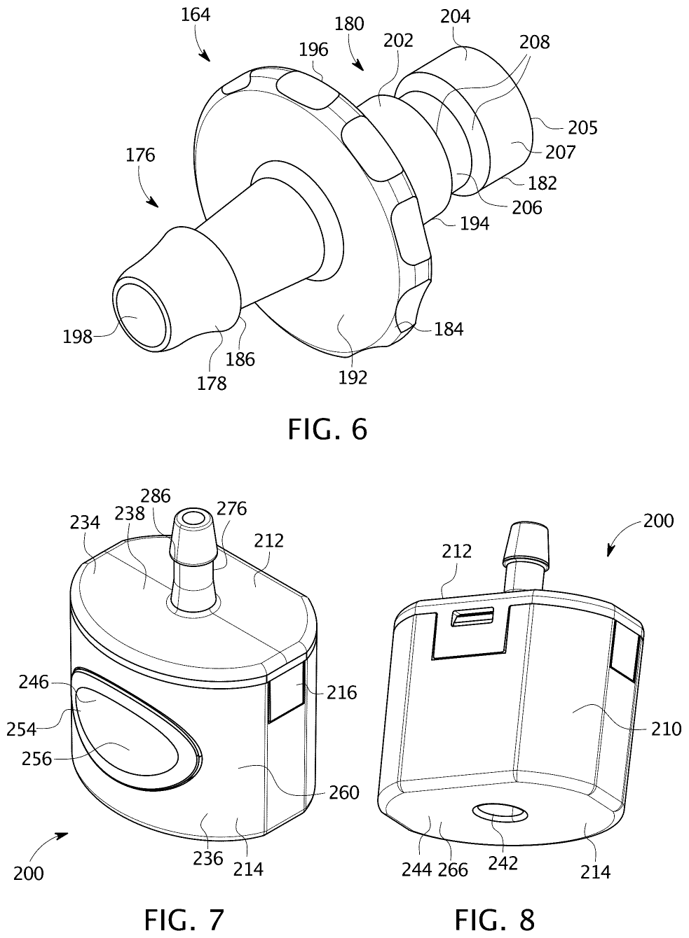

As illustrated in FIG. 6, embodiments of a connector 164 may comprise a first end 176 comprising a barbed connector portion 178 and a second end 180 comprising a slotted connector portion 182. The connector 164 may comprise a collar 184 positioned between the two ends and extending radially. As illustrated, for example, in FIG. 4, the barbed connector portion 178 may be inserted into a lumen 160 of the tube 158 such that a barb 186 of the connector engages an inside surface 188 of the lumen and secures the connector 164 to the tube. The barbed portion 176 may be inserted into the lumen 160 until and end 190 of the tube contacts a surface 192 of the collar 184.

The slotted connector portion 182 of the connector 164 may comprise a generally cylindrical extension 194 extending from an opposite surface 196 of the collar 184 and surrounding a central bore 198 of the connector. The central bore 198 may be generally circular, comprising a central axis. The cylindrical extension 194 may comprise a base portion 202 and a tip portion 204 separated by a slot 206 that extends radially inwardly from an outer diameter of the cylindrical extension. The tip portion may extend between the slot 206 and a tip 205. The slot may create shoulders 208 on either side of the slot. The slot may extend around the entire circumference of the extension 194, or it may comprise one or more slots that extend only around a portion of the circumference. The slotted connector portion 178 of the connector 164 may comply with the standards set forth in IEC 80369-5: 2016, “Small-bore connectors for liquids and gases in healthcare applications—Part 5: Connectors for limb cuff inflation applications,”which is hereby incorporated by reference.

Blood pressure cuffs and blood pressure monitors may use a variety of tube configurations, including single tube connections and dual tube connections. FIGS. 7-77 show illustrative embodiments of adapters that allow for the attachment of various cuff tube configurations with various monitor tube configurations.

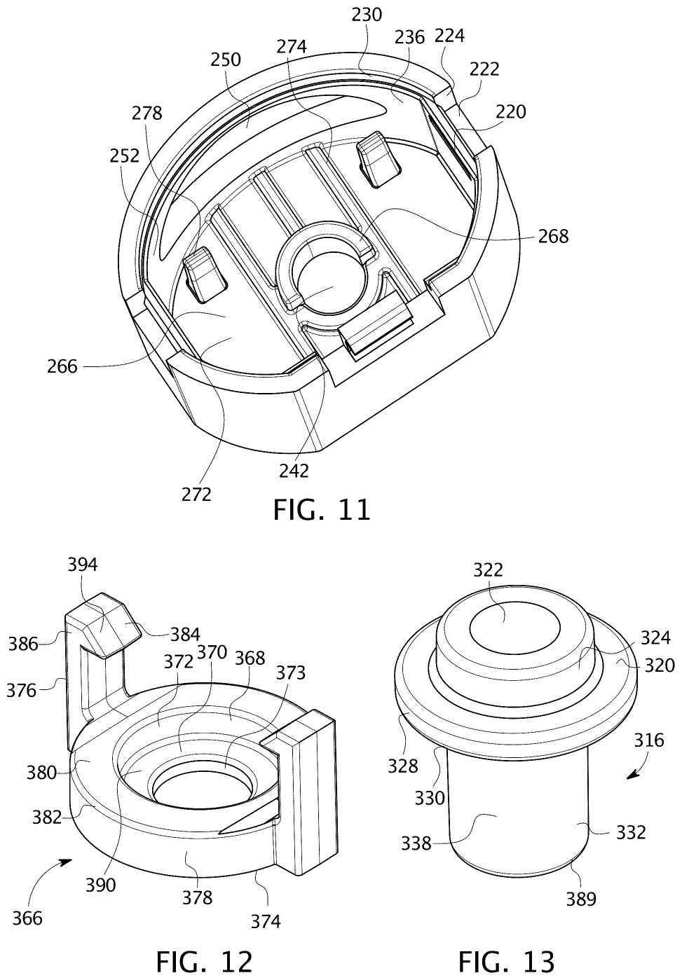

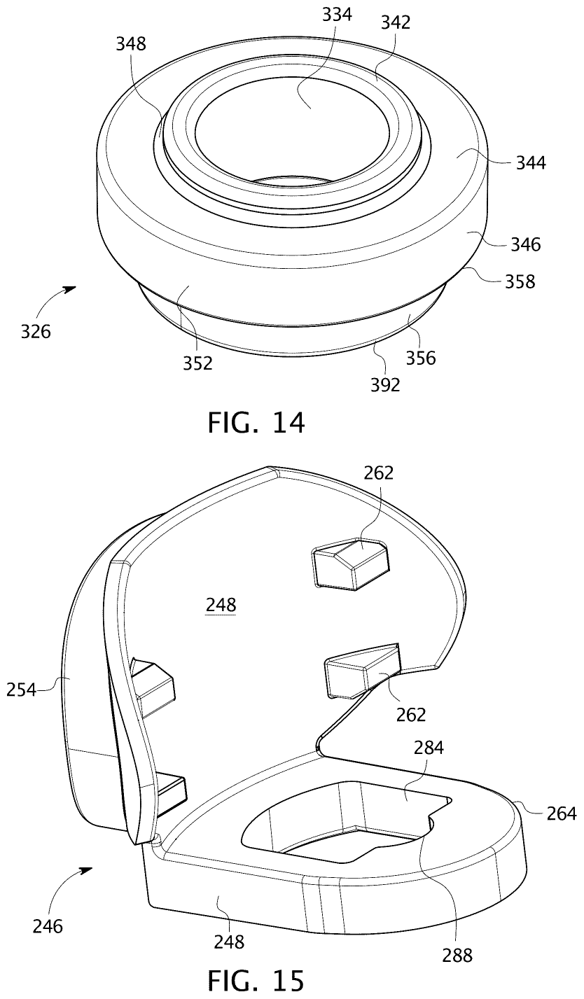

FIGS. 7-15 illustrate embodiments of an adapter 200 that enables releasable connection of a male slotted connector 182 with a single tube, such as tube 58 (FIG. 2). For example, the adapter 200 may be connected to a tube 58 extending from a monitor 42. A single tube may extend from a cuff and terminate in a connector 164. The adapter may, accordingly, allow releasable connection of the cuff and connector.

The adapter 200 may comprise a case 210. The case may comprise a top case 212 and a bottom case 214. The top and bottom cases may snap together. In particular, the top case 212 may comprise one or more arms 216 comprising tapered tabs 218. The bottom case 214 may comprise one or more slots or apertures 220 sized and adapted to receive the tabs 218. Top case arms 216 may be flexible and resilient such that the tabs 218 snap into place in the apertures 220 and lock the top case 212 and bottom case 214 together when the adapter is assembled. The bottom case 214 may comprise cut-outs 222 that accommodate a portion of the top case arms 216. The top case arms and bottom case cut-outs may have mating surfaces 224, 226 that abut one another to align the top and bottom cases relative to one another. The top case 212 may also comprise an extensions or rib 228 that extend from a lower surface 232 of the top case lid 234. A portion of rib 228 may engage a corresponding inside surface 230 of bottom case sidewall 236. Interlocking portions of the top case 212 and bottom case 214 may be reversed such that, for example, the arms extend from the bottom case and engage apertures in the top case.

A barbed connector 276 may extend from a top surface 238 of the top case lid 234. The barbed connector 276 may be inserted into a lumen of the tube, for example connect to a blood pressure monitor, such that a barb 286 of the connector engages an inside surface of the lumen and secures the adapter 200 to the tube. A bore 298 may extend through the barbed connector into a cavity formed within the adapter case 210. The bottom case 214 may comprise an aperture 242 that extends through a bottom surface 244. The aperture may be generally circular and sized to accommodate a slotted connector. The aperture may extend into the cavity formed with the adapter case 210. An internal rim 268 may extend upwardly from an interior surface 272 of the bottom case bottom wall 266. The rim 268 may extend adjacent to a portion of the aperture 242. For example, the rim 268 may form a semi-circle, extending half-way around the aperture 242. Ribs 274 may extend upwardly from the interior surface 272. In addition, one or more stops 273 may also extend upwardly from the interior surface 272. The stops may be higher than the ribs 274.

The adapter 200 may comprise a connecting and sealing mechanism that allows the adapter 200 to releasable connect to a slotted connector (182). The adapter may comprise a button 246. The button may comprise an actuation portion 248 that extends generally along, parallel, or adjacent to an inside surface 252 of the bottom case sidewall 236. The case sidewall 236 may be curved and the actuation portion of the button 246 may be correspondingly curved. A pad 254 may extend from the actuation portion 248. The pad may extend outwardly or perpendicularly to the actuation portion. The pad 254 may extend through an aperture 250 extending through the bottom case sidewall 236. An outside surface 256 of the pad 254 may generally match the contour of an outside surface 260 of the bottom case sidewall 236. One or more tabs 262 may extend inwardly from the actuation portion. The tabs may aid with alignment and linear motion of the button 246.

The button may comprise a key portion 264 that extends from the actuation portion 248. The key portion may extend perpendicularly from the actuation portion. The key portion 264 may extend generally parallel to the bottom case bottom wall 266. A lower surface 280 of the key portion 264 may abut an upper surface 282 of the rib 274. The key portion may include a cut-out 284 that extends through the key portion. The cut-out 284 may comprise an edge portion 288. The edge portion may be concave or semi-circular and may generally mirror the internal rim 268 of the bottom case 214. The thickness 290 of the key portion may be smaller than the slot (206) that is formed in the slotted connector (182).

An extension 240 may extend downwardly from the lower surface 232 of the lid 234. The extension 240 may comprise a generally cylindrical shape, having cylindrical inner 292 and outer 294 sidewalls, with the inner sidewall forming a cavity 354. The extension may be closed in part at an upper end 296 by the lid lower surface 232 and open at a lower end 302. The inner sidewall 292 may comprise a first portion 304 having a smaller diameter and a second portion 306, closer to the lower end 302, having a larger diameter such that a step or shoulder 308 is formed between the portions.

A spring 310 or other biasing element may be inserted into the extension 240. A first end 314 of the spring may seat against the lower surface 232, and a rib or tab 312 may extend from the lower surface to position the first end 314 of the spring. The first portion 304 of the extension sidewall may surround and contain the spring.

The adapter may also comprise an ejector 316 that abuts a lower end 318 of the spring 310. The ejector may comprise a through bore 322, a collar 328 forming an upper shoulder 320, and an upper cylindrical portion 324 extending upwardly from the shoulder. The spring lower end 318 may seat against the shoulder 320, surrounding the cylindrical portion 324 such that lateral movement of the spring is restrained, and the spring asserts a force against between the lid 234 and the ejector 316 biasing the ejector away from the lid. The ejector may further comprise a lower protrusion 332 that extends downwardly from a lower shoulder 330. The protrusion may be generally cylindrical The adapter may further comprise a retainer 326. The retainer comprises a through bore 334. The bore may have a diameter that is sufficient that the lower protrusion 332 of the ejector 316 can be inserted into the bore 334 of the retainer. An inside surface 336 of the retainer bore 334 and an outside surface 338 of the ejector lower protrusion 332 may have complementary shapes such that a uniform gap 340 separates the two pieces when the adapter is assembled. The retainer collar 346 may comprise a generally cylindrically shaped exterior surface 352. The outer surface 352 of the retainer may have a diameter that is smaller than and has a complementary shape to the lid extension second portion 306. Accordingly, when the adapter is assembled, the retainer can be at least partially inserted into the second portion 306 of the extension. The diameter of the retainer collar outer surface may be larger than the lid extension first portion 304 to that the step 308 prevents the retainer from insertion deeper into the extension cavity 354.

The retainer may have an upper protrusion 342 extending upwardly from an upper surface 344 of a collar 346. The upper protrusion 342 and upper surface 344 may intersect to form a step 348. When the adapter is assembled, an O-ring 350 may be positioned between the step 348 of the retainer 326 and the step 308 of the lid extension 240 such that fluid flow is restricted or an air-tight seal is formed between the retainer and the lid. The retainer may also comprise a lower protrusion 356 that extends downwardly from a lower surface 358 of the collar 346. A counterbore 360 may extend upwardly into the lower protrusion 356 surrounding the through bore 334 such that a shelf or seat 362 is formed. When the adapter is assembled, an O-ring 364 may be positioned between the seat 362 of the retainer and the outer surface 338 of the ejector lower protrusion 332. The O-ring 364 may be spaced apart from the outer surface 338 of the ejector or may contact the outer surface such that fluid flow is restricted or an air-tight seal is formed between the retainer 326 and the ejector 316.

The adapter may further comprise a cap 366. The cap may comprise a through bore 368. A rim 370 may extend inwardly from an inner surface 372 of the through bore. The rim may have a generally cylindrical inner wall 373 that may have a diameter that is sufficient that the lower protrusion 332 of the ejector 316 can be inserted into the rim 370 of the cap. An inside surface 373 of the cap rim 370 and an outside surface 338 of the ejector lower protrusion 332 may have complementary shapes such that a uniform gap separates the two pieces when the adapter is assembled such that the ejector can traverse up and down within the rim 370 of the cap 366 and the bore 334 of the retainer 326. When assembled, a lower surface 374 of the cap may be seated against a portion of an upper surface of the bottom case internal rim 268.

Arms 376 may extend from an outer surface 378 of the cap. The arms may extend upwardly above a top surface 380 of the cap body 382. Embodiments of the cap may comprise two arms positioned on opposite sides of the cap body. The arms may comprise angled tabs 384 extending inwardly from end portions 386 of the arms that are spaced apart from the cap top surface 380. The tabs 384 may engage complimentary slots 388 formed in the outer sidewall 294 of the lid extension 240 when the adapter is assembled. Other numbers of arms and slots or other configurations for the slots could be used. The arms 376 and tabs 384 may secure the upper surface 380 of the cap against the lower end 302 of the lid extension sidewall 294. The cap upper surface 380 may contact or abut the lower surface 358 of the retainer collar 346 and an upper surface 390 of the cap rim 370 may contact or abut a lower end 392 of the retainer lower protrusion 356. The arms may be formed of resilient material such that an angle portion 394 of the arm tabs 384 pushes the end of the arm outward when the cap is assembled over the lid extension 240, and the tabs snap into place in the extensions slots 388 when the cap reaches a fully assembled position.

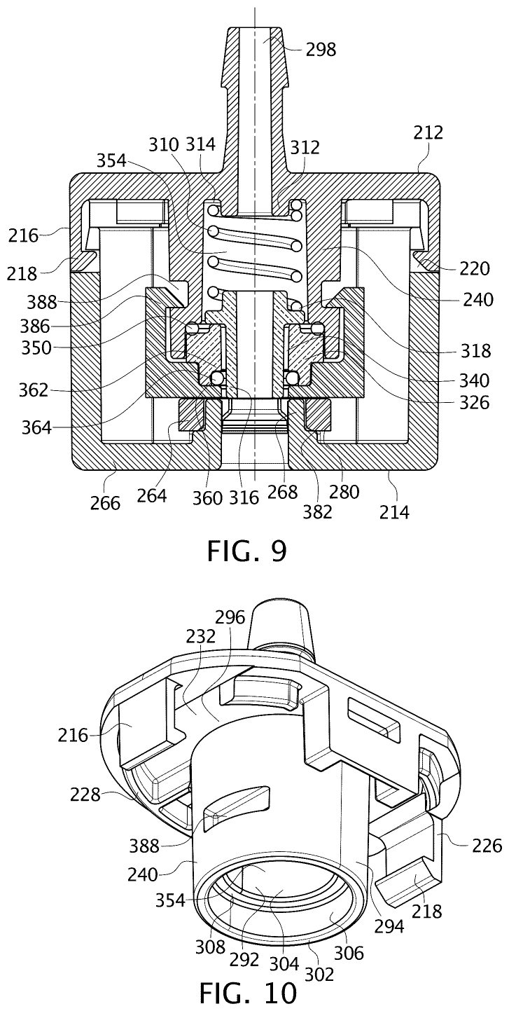

As illustrated in FIGS. 7-15, and particularly with reference to FIG. 9, the adapter 200 may be snapped together in such a manner that the ejector 316 is biased in a down position by the spring 310 but is constrained from extending beyond the downward position by the retainer 326 and cap 366. In an embodiment of an assembly process, a top case 212 having a lid is provided with a barb connector 278 extending from a top surface and a cylindrical extension 240 extending from a lower surface. A spring 310 is inserted into a cavity of the extension 240. The spring may have a spiral, helix, or cylindrical shape, and the spring is positioned such that the upper end of the spring engages a tab 312 extending from the lid lower surface. An ejector 316 is then inserted into the cavity 354 below the spring. A portion of the ejector is positioned in the cavity and a portion extends beyond a lower end of the cavity. The spring presses against an upper surface of the ejector biasing the ejector downwardly and in a direction out of the extension cavity. A retainer 326 is inserted at least partially into the cavity after the ejector. The retainer allows a lower portion of the ejector to pass through a bore through the retainer, but an upper surface of the retainer applies a force against a portion of the ejector to counter act the force of the spring and restrain movement of the ejector within a certain linear range. A cap is then snapped into place on the lid extension and below the retainer. The cap is secured to the extension, and an upper surface of the cap provides a force against a lower surface of the retainer to help hold the retainer in place. A button 246 is positioned in a bottom case 214. The button has an aperture that surrounds portion of the ejector extending below the retainer and cap. The ejector lower portion may extend into an aperture formed in the bottom case. The bottom case is snapped into place on the top case, holding the entire assembly together.

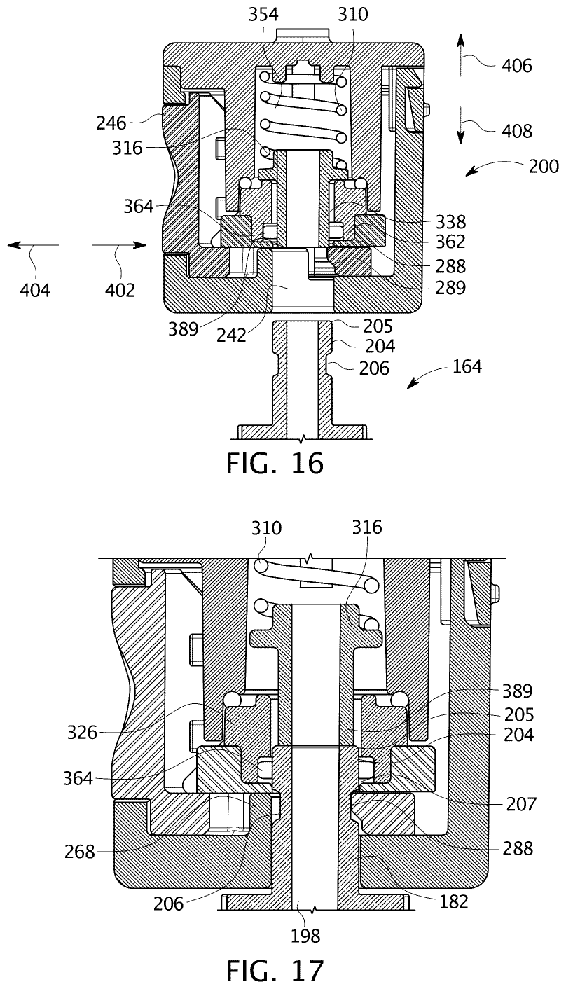

Operation of an embodiment of the adapter will be discussed with reference to FIGS. 16-17. FIG. 16 shows the adapter 200 and connector 164 in an initial, unconnected state. FIG. 17 shows the adapter and connector in a subsequent, connected state. The button 246 is linearly movable in retraction 402 and extension 404 directions between extended and retracted positions. In the initial state, the button is in the extend position, and the button is biased toward the extended position. The button biasing force may be provided by contact between one or more button tabs 262 and one or more bottom case stops 273. The tabs and/or stops may be made from resilient material to provide a spring or biasing force that returns the button to the extended position when it is released. In the extended position, a portion of the button edge 288 occludes a portion of the bottom case aperture 242. As the connector is moved into the adapter in an upward direction 406, the tip 205 of the connector is pressed into the adapter, the tip contacts a sloped portion 289 button edge 288. The connector tip forces the button in the retraction direction 404 moving the concave edge 288 out of the path of the connector. As the connector is further inserted into the adapter, the connector tip 205 contacts the lower end 389 of the ejector and moves the ejector in the upward direction 406 against the bias of the spring 310. As the connector is fully inserted into the adapter, the button edge 288 moves past the connector tip portion 204 to the slot 206. The biasing force of the button moves the button in the extension direction 404, and the button edge 288 engages the connector slot 206. The engagement between the slot 206 and button edge 288 secures the connector from moving in a downward direction 408 and connector is secured to the adapter.

A connector that has been fully inserted into the adapter and engaged by the button may be released by pressing the button in the retraction direction 402. As the button is pressed in the retraction direction 402 against its bias, the button edge 288 moves out of the connector slot 206, and the biasing force of the spring 310 presses the lower end 389 of the ejector against the tip 205 of the connector, forcing the connector to move in a downward direction 408 out of the adapter aperture 242. Alternatively, a spring 310 and ejector 316 may be omitted, and the connector may be removed by the user manually applying a force in the downward direction to remove the connector from the adapter.

Additionally, when the adapter is in the unconnected condition, the ejector 316 is bias in the downward direction 408, and the O-ring 364 is positioned between the outside surface 338 of the ejector lower portion 332 and the corresponding seat 362 formed on the retainer 326. When the adapter is in the connected condition, the ejector 316 is bias in the upward direction 406, and the O-ring 364 is positioned between the outside surface 207 of the connector tip portion 204 and the corresponding seat 362 formed on the retainer 326. Accordingly, a sealed fluid path may be created between the cavity 354 and the central bore 198 of the connector.

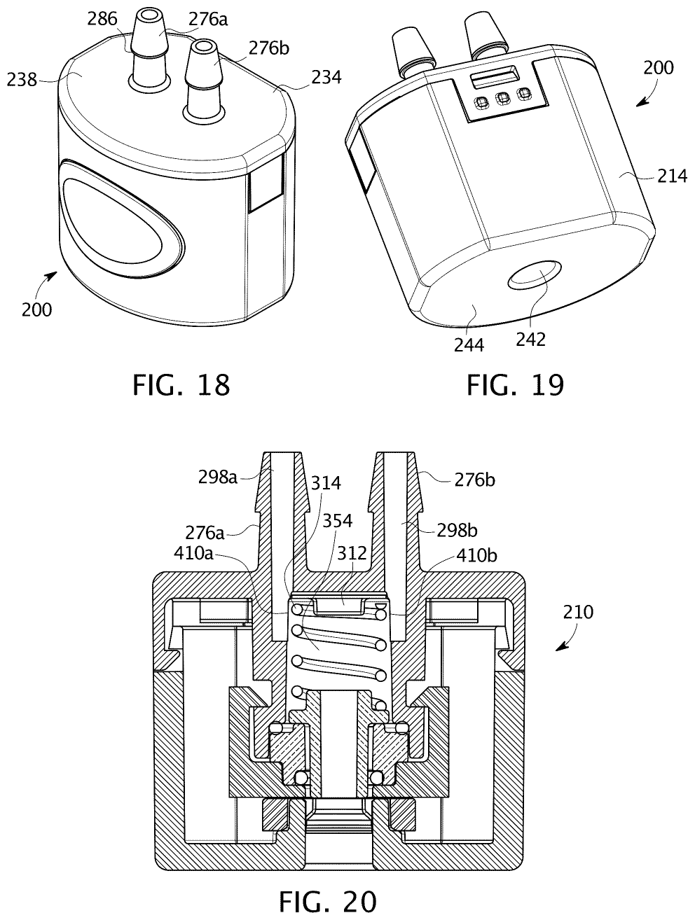

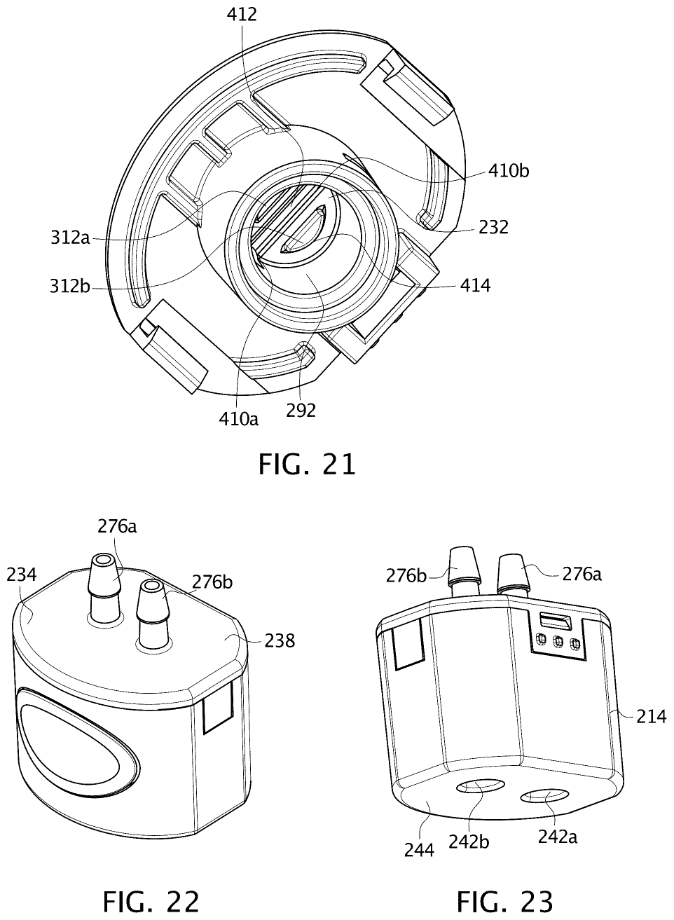

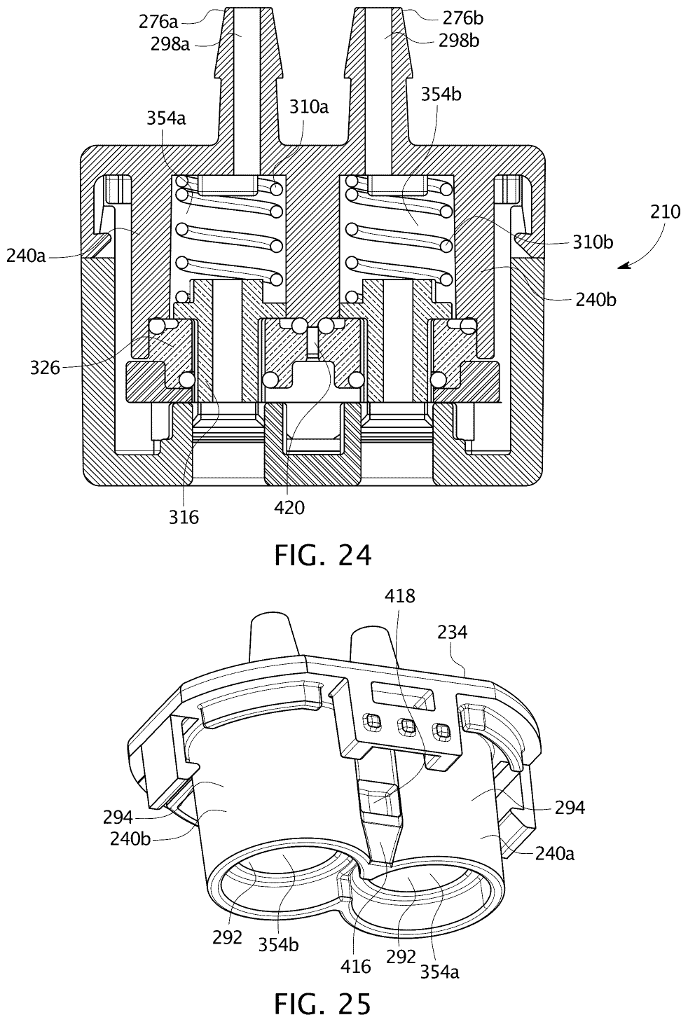

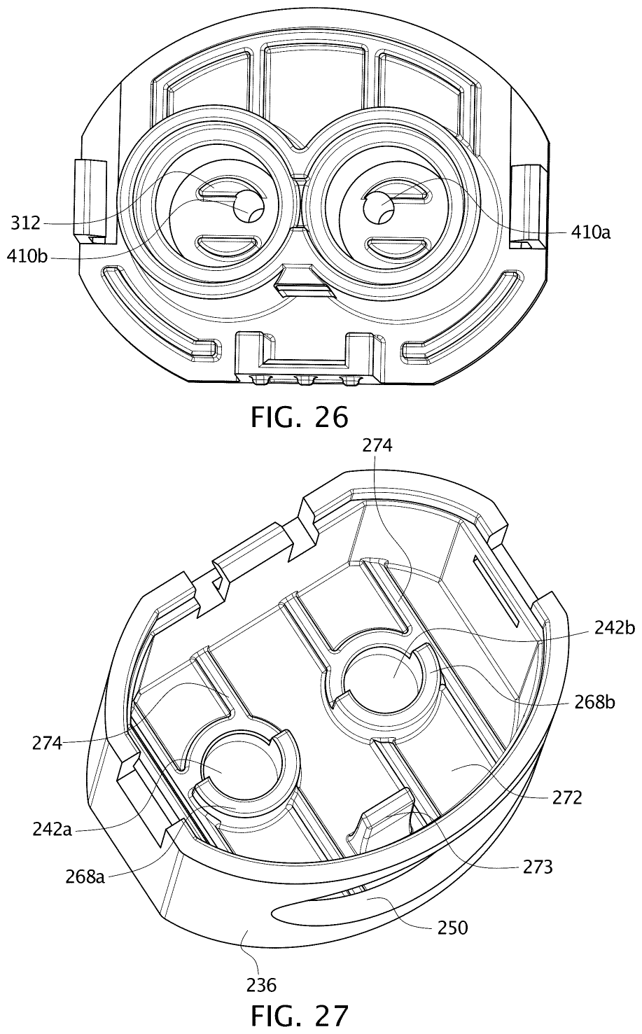

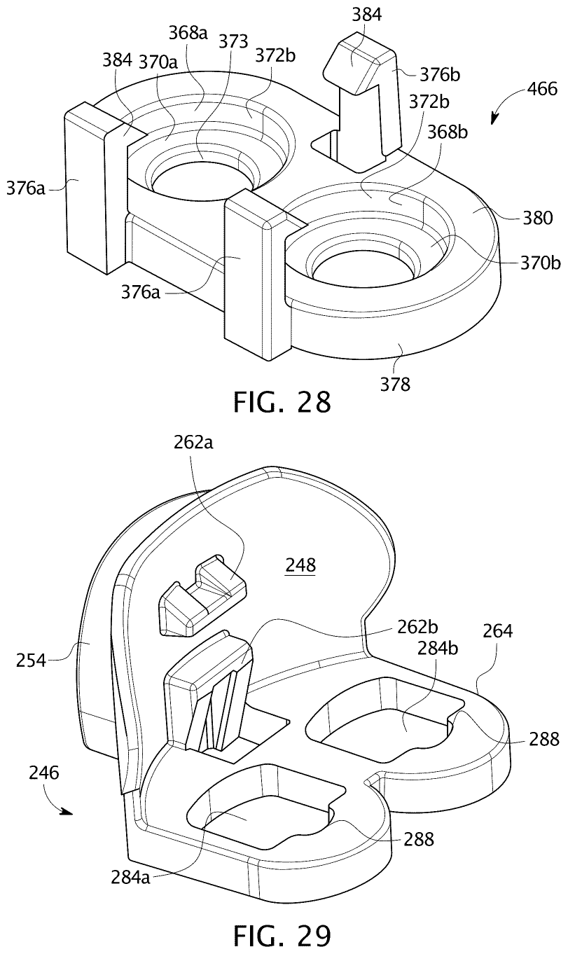

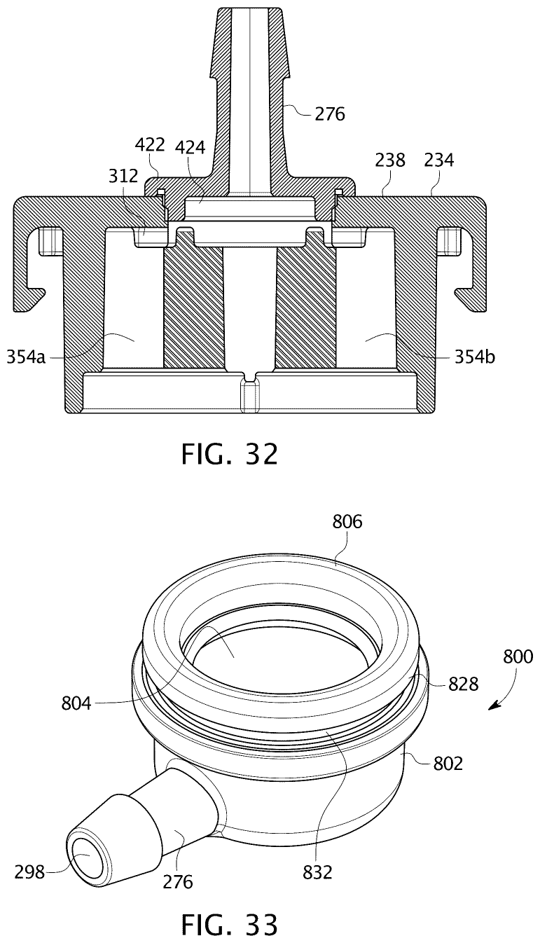

FIGS. 7-15 and the accompanying description above illustrate embodiments of an adapter having one barbed connector and one aperture for inserting a connector. Alternative embodiments of the adapter may comprise more than one barbed adapter and/or more than one aperture for connection to multiple connectors or a multi-prong connector (see, for example, FIG. 4). Embodiments of such alternatives are illustrated in FIGS. 18-32 and described below. Certain elements that differ from single barb/connector embodiments are discussed, but one of ordinary skill in the art would understand that other elements of multi-barb/connector embodiments are the same or readily derivable from the description of single barb/connector embodiments or the understanding of one having ordinary skill, and a description of such elements may not be repeated or provided.

FIGS. 18-21 illustrate embodiments of an adapter 200 comprising a single aperture 242 extending through the bottom surface 244 of the bottom case 214. The embodiments comprise first 276a and second 276b barbed connectors extending from the top surface 238 of the case lid 234.

The barbed connectors 276 may each be inserted into a lumen of the tube, for example connect to a blood pressure monitor, such that a barb 286 of the connector engages an inside surface of the lumen and secures the adapter 200 to the tube. A first bore 298a may extend through the first barbed connector 276a connected to a cavity 354 formed within the adapter case 210. An aperture 410a may be formed in the inner sidewall 292 connecting the first bore 298a with the cavity 354. A second bore 298b may extend through the second barbed connector 276b into the cavity 354. A second aperture 410b may be formed in the inner sidewall 292, which may be opposite the first aperture 410a, connecting the second bore 298b with the cavity 354. A channel 412 may extend across the lower surface 232 of the lid between the first 410a and second 410b apertures. A rib or tab 312 may extend from the lower surface 232 to position the first end 314 of the spring. The tab 312 may comprise a first tab 312a and a second tab 312b. The tabs 312a, 312b may be positioned on opposite sides of the channel 412. The tabs may comprise semi-circular sections such that the outer edge 414 of the tab generally conforms to the circular inside diameter of the spring. The bottom case 214 may comprise an aperture 242 that extends through a bottom surface 244.

FIGS. 22-29 illustrate embodiments of an adapter 200 comprising first 242a and second 242b apertures extending through the bottom surface 244 of the bottom case 214. The embodiments comprise first 276a and second 276b barbed connectors extending from the top surface 238 of the case lid 234. The barbed connectors 276 may each be inserted into a lumen of the tube, for example connect to a blood pressure monitor, such that a barb 286 of the connector engages an inside surface of the lumen and secures the adapter 200 to the tube. A first bore 298a may extend through the first barbed connector 276a connected by a first aperture 410a to a first cavity 354a formed within the adapter case 210. A second bore 298b may extend through the second barbed connector 276b connected by a second aperture 410b to a second cavity 354b formed within the adapter case 210. First and second cavities 354a, 354b may be fluidly isolated or may be connected through an aperture 420 between the cavities.

The bottom case 214 may comprise a first aperture 242a and a second aperture 242b that extend through a bottom surface 244. The apertures may be generally circular and sized to accommodate a slotted connector. The aperture may extend into the cavities formed within the adapter case 210. An internal rim 268a, 268b may extend upwardly from an interior surface 272 of the bottom case bottom wall adjacent each aperture. Ribs 274 may extend upwardly from the interior surface 272. In addition, one or more stops 273 may also extend upwardly from the interior surface 272. The stop may be higher than the ribs 274.

An extension 240 may extend downwardly from the lower surface 232 of the lid 234. The extension 240 may comprise a first extension 240a and a second extension 240b may each comprise a generally cylindrical shape, having cylindrical inner 292 and outer 294 sidewalls, with the inner sidewall forming cavities 354a, 354b. Outer sidewalls 294 may intersect such that the outside of the extensions form overlapping circles and a web 416 may extend between the sidewalls. A slot 418 may be formed in the web. Slots may be formed in the outer sidewalls 294 opposite the web slot 418.

A spring 310a, 310b or other biasing element may be inserted into the extension 240. A first end 314 of the spring may seat against the lower surface 232, and a rib or tab 312 may extend from the lower surface to position the first end 314 of the spring. An ejector 316 and retainer 326 may be positioned in each extension cavity 354a, 354b.

The adapter may further comprise a cap 466. The cap may comprise a first through bore 368a and a second through bore 368b. A rim 370a, 370b may extend inwardly from each inner surface 372a, 372b of the through bore. The rim may have a generally cylindrical inner wall 373 that may have a diameter that is sufficient that the lower protrusion 332 of the ejector 316 can be inserted into the rim 370 of the cap. An inside surface 373 of the cap rim 370 and an outside surface 338 of the ejector lower protrusion 332 may have complementary shapes such that a uniform gap separates the two pieces when the adapter is assembled such that each ejector can traverse up and down within the rim 370 of the cap 366 and the bore 334 of the retainer 326.

Arms 376a, 376b may extend from an outer surface 378 of the cap. The arms may extend upwardly above a top surface 380 of the cap body 382. Embodiments of the cap may comprise two arms 376a positioned on first side of the body to engage slots formed in extension sidewalls 294 and a single arm 376b on an opposite side of the cap body to engage the slot 418 formed in web 416. The arms may comprise angled tabs 384 extending inwardly from end portions of the arms.

The adapter may comprise a button 246. The button may comprise an actuation portion 248 that extends generally along, parallel, or adjacent to an inside surface of the bottom case sidewall. A pad 254 may extend from the actuation portion 248. The pad may extend outwardly or perpendicularly to the actuation portion. The pad 254 may extend through an aperture 250 extending through the bottom case sidewall 236. One or more tabs 262a, 262b may extend inwardly from the actuation portion. The tabs may aid with alignment and linear motion of the button 246.

The button may comprise a key portion 264 that extends from the actuation portion 248. The key portion may extend perpendicularly from the actuation portion. The key portion 264 may extend generally parallel to the bottom case bottom wall 266. The key portion may include a cut-outs 284a, 284b that extend through the key portion. Each cut-out may comprise an edge portion 288. The edge portion may be concave or semi-circular and may generally mirror the internal rims 268a, 268b of the bottom case 214.

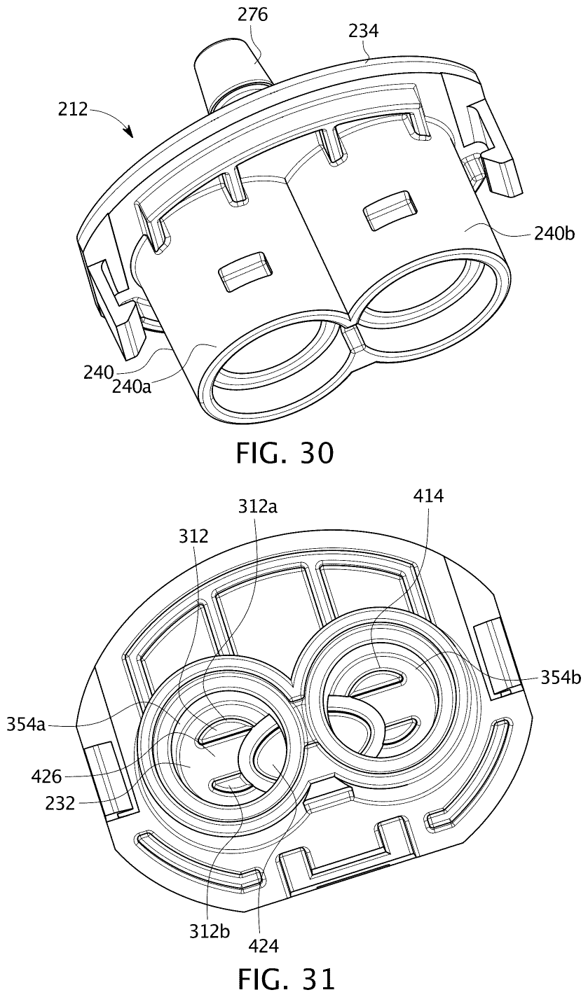

FIGS. 30-32 illustrate embodiments of a top case 212 for use with an adapter comprising first 242a and second 242b apertures extending through the bottom surface 244 of the bottom case 214 (see FIG. 23). The embodiments comprise a single barbed connector 276 extending from the top surface 238 of the case lid 234. The barbed connector may be integrally formed with the case lid, or it may comprise an insert 422 that is positioned in an aperture in the case lid. A recess or channel 424 may extend across the lower surface 232 of the lid between the first 354a and second 354b cavities. A rib or tab 312 may extend from the lower surface 232 to position the first end 314 of the spring. The tab 312 may comprise a first tab 312a and a second tab 312b. The tabs 312a, 312b may be positioned on opposite sides of a channel 426. The first tab 312a may comprise a semi-circular section such that the outer edge 414 of the tab generally conforms to the circular inside diameter of the spring. The second tab 312b may comprise a truncated form of the semi-circle where it intersects with the channel 424. An extension 240 may extend downwardly from the lower surface 232 of the lid 234. The extension 240 may comprise a first extension 240a and a second extension 240b may each comprise a generally cylindrical shape, having cylindrical inner 292 and outer 294 sidewalls, with the inner sidewall forming cavities 354a, 354b.

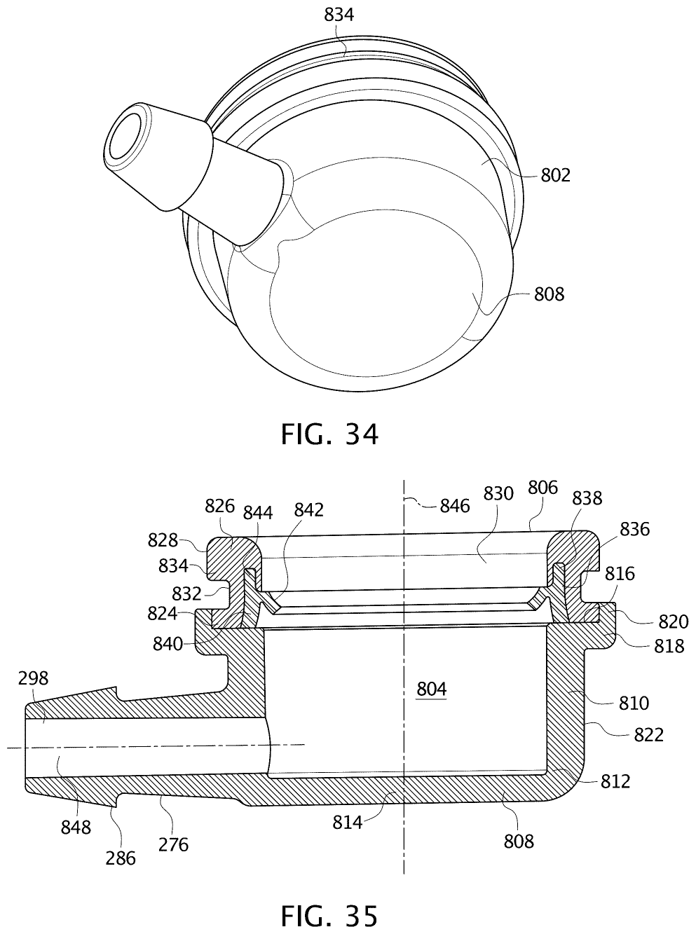

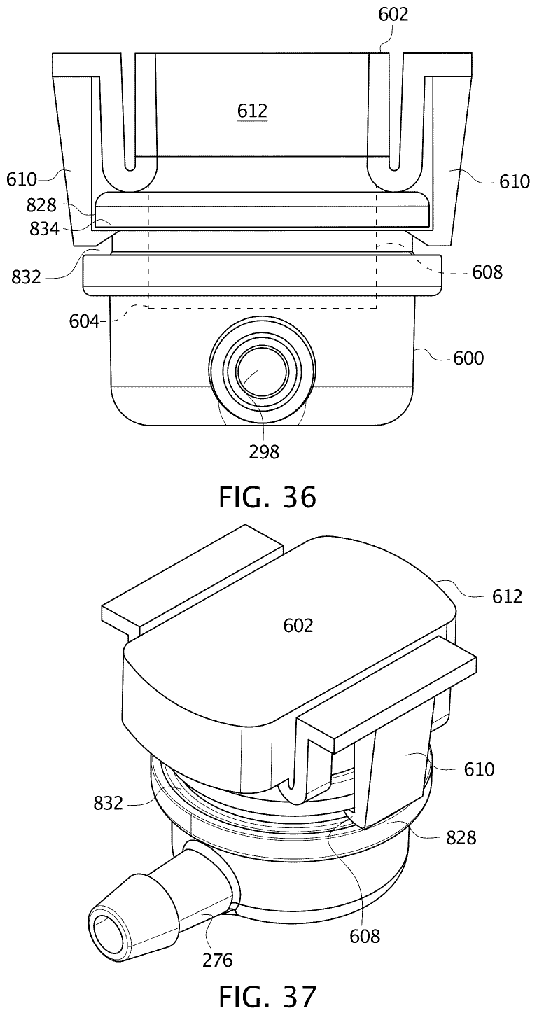

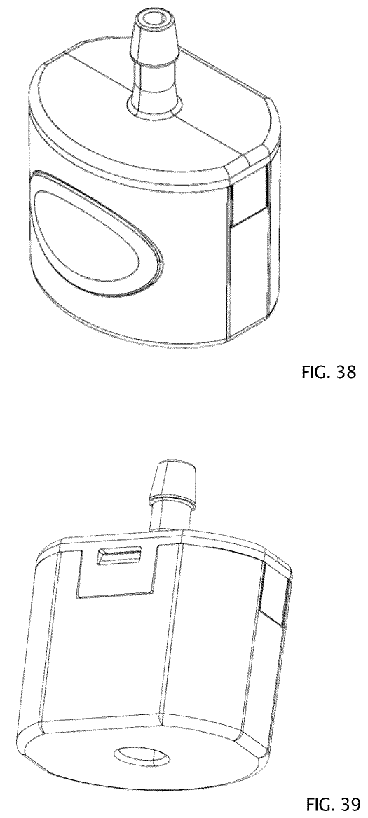

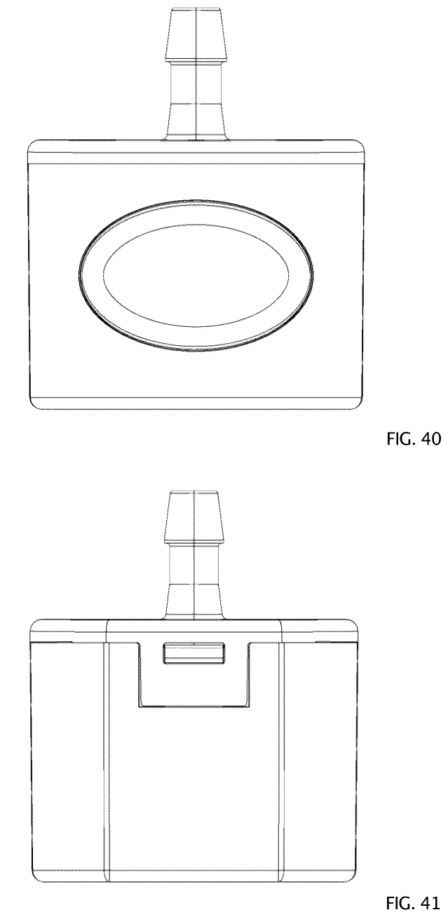

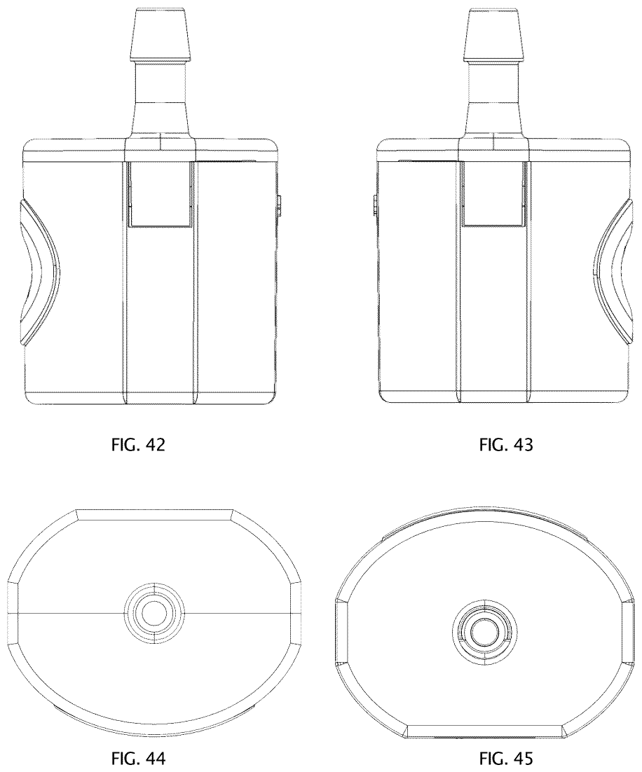

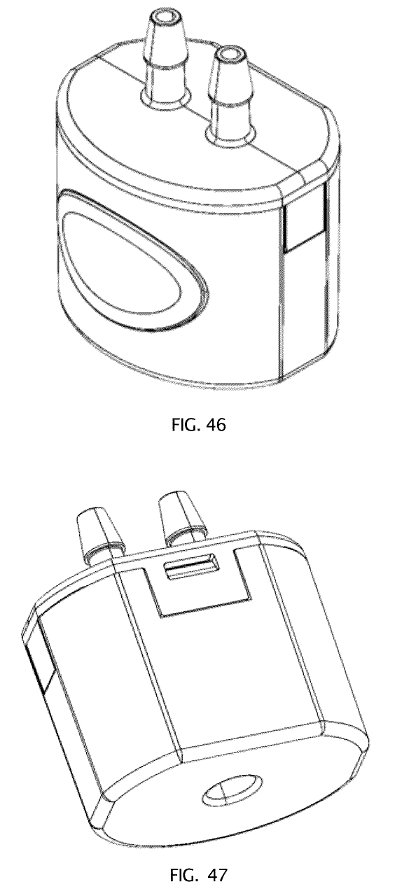

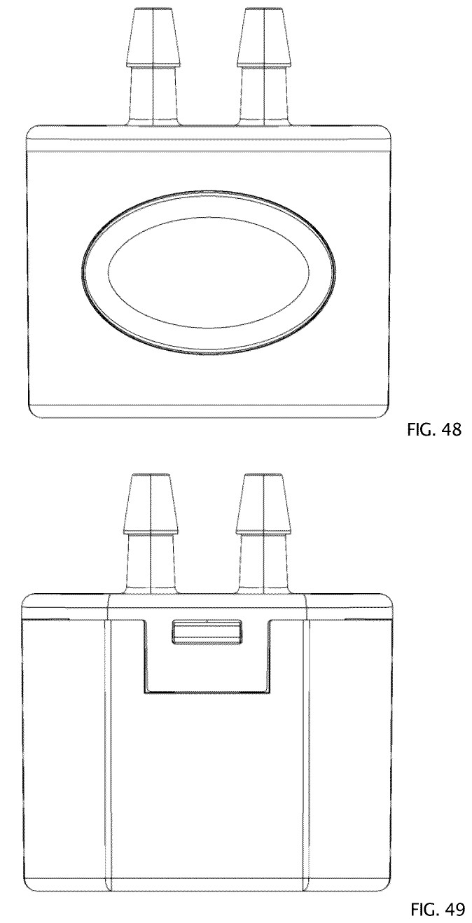

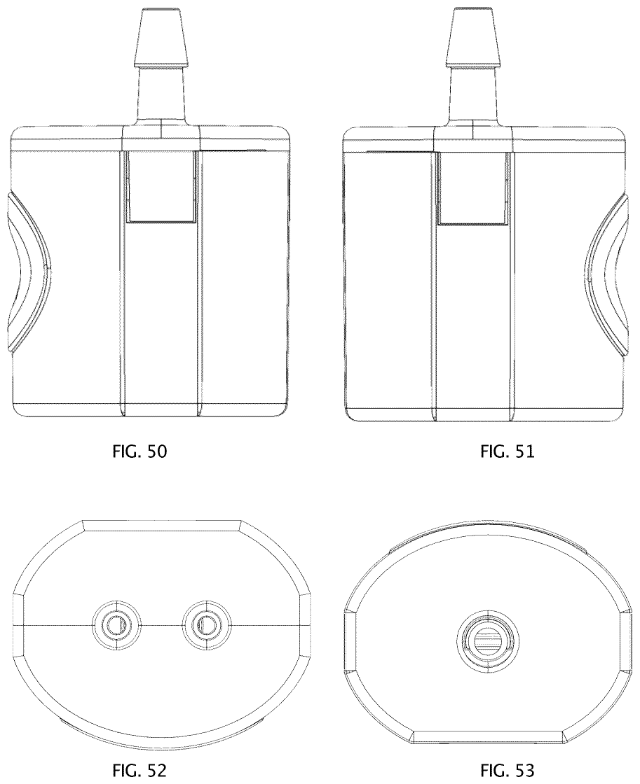

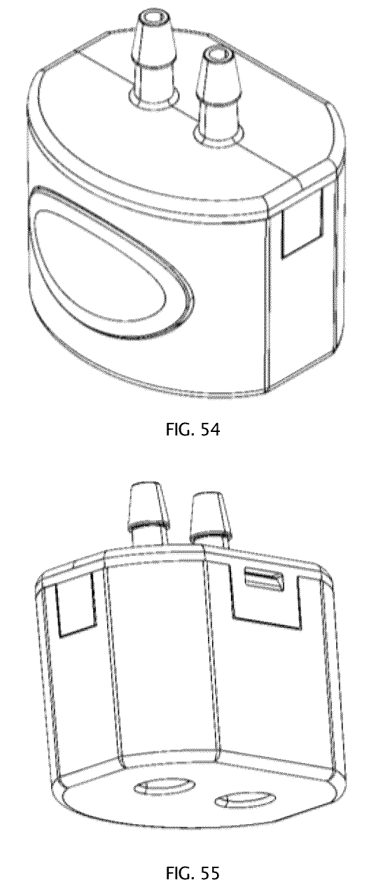

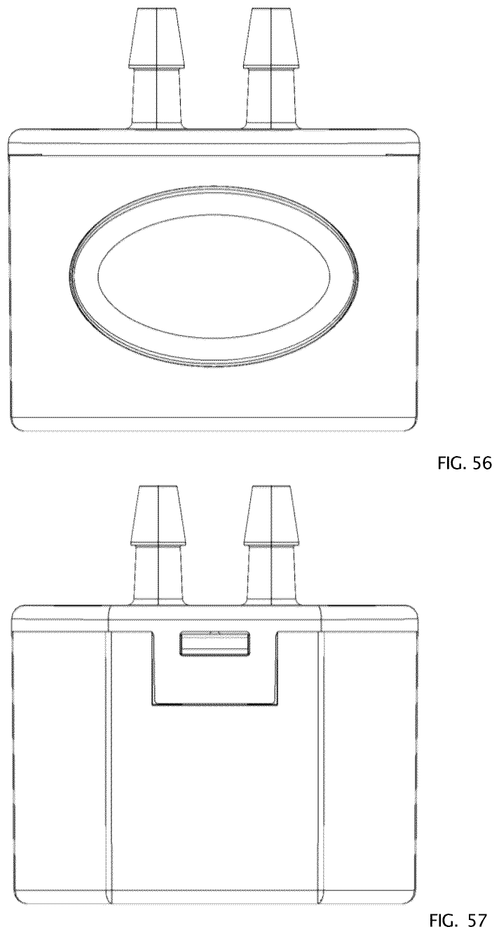

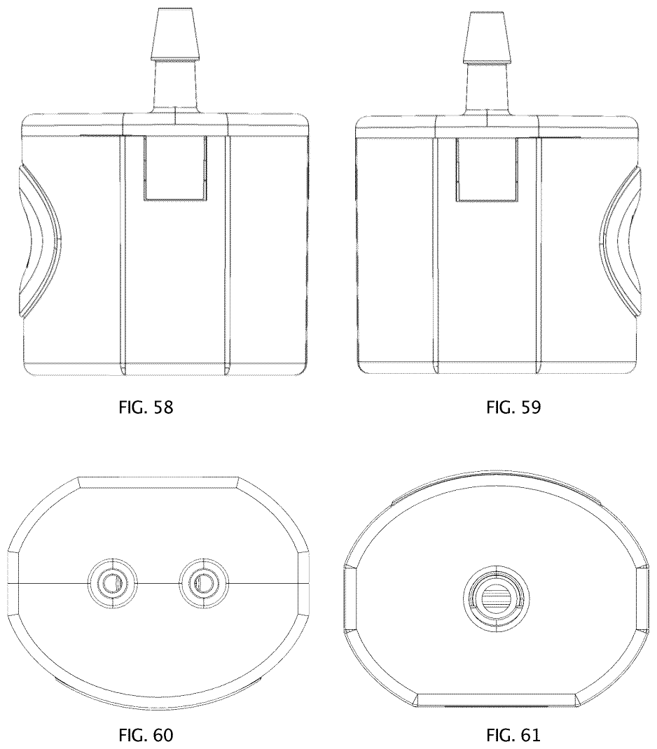

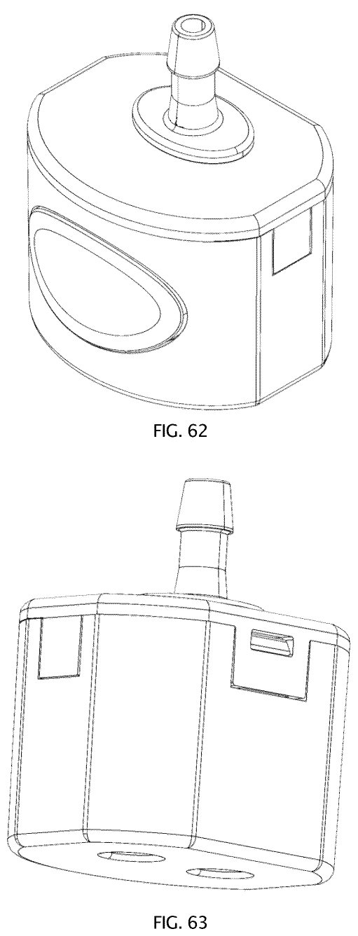

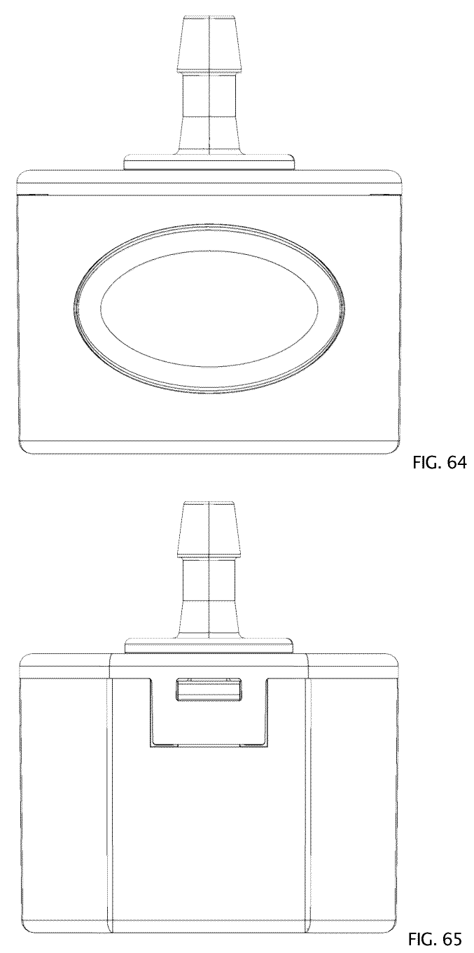

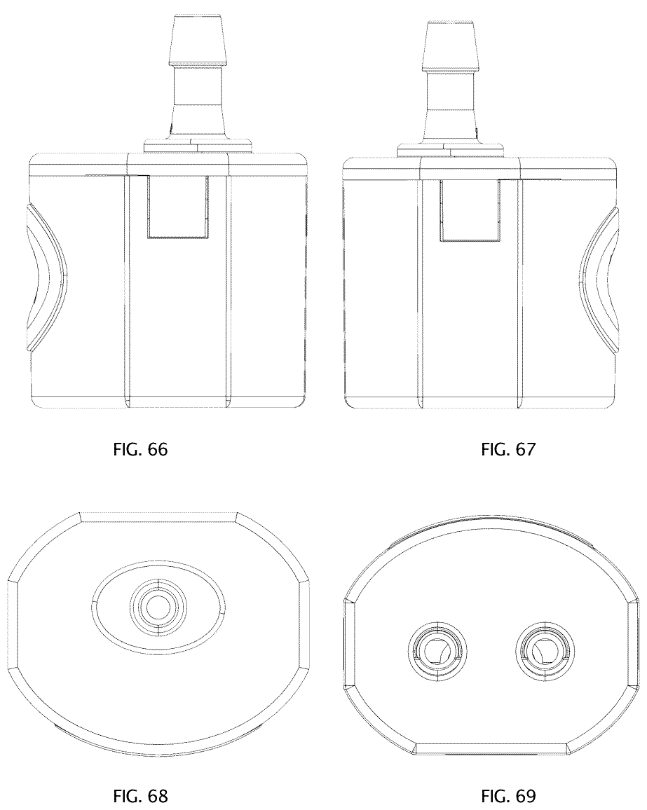

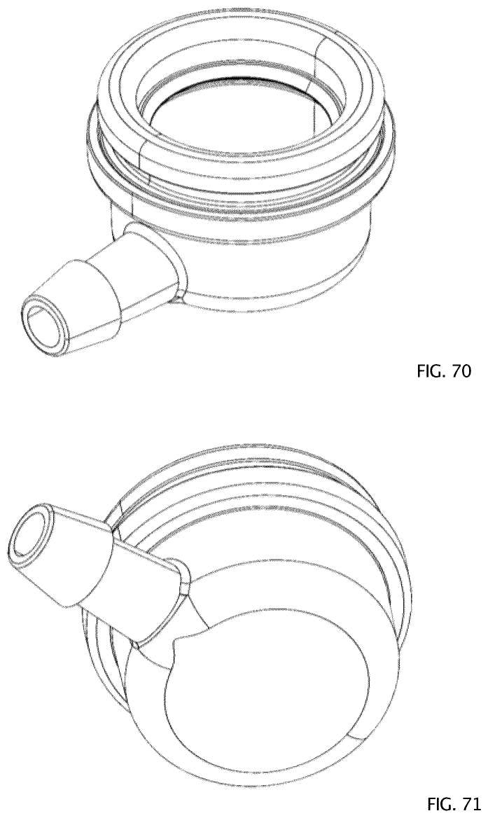

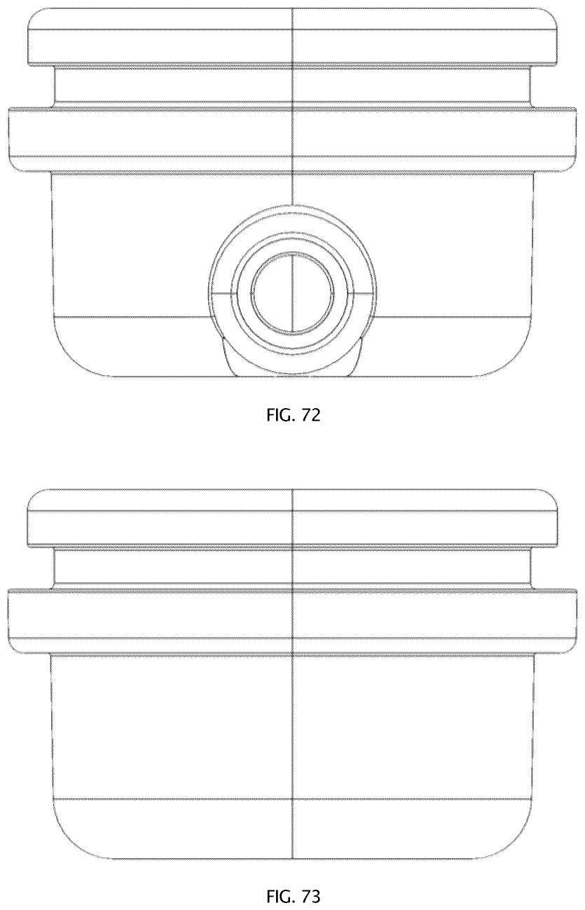

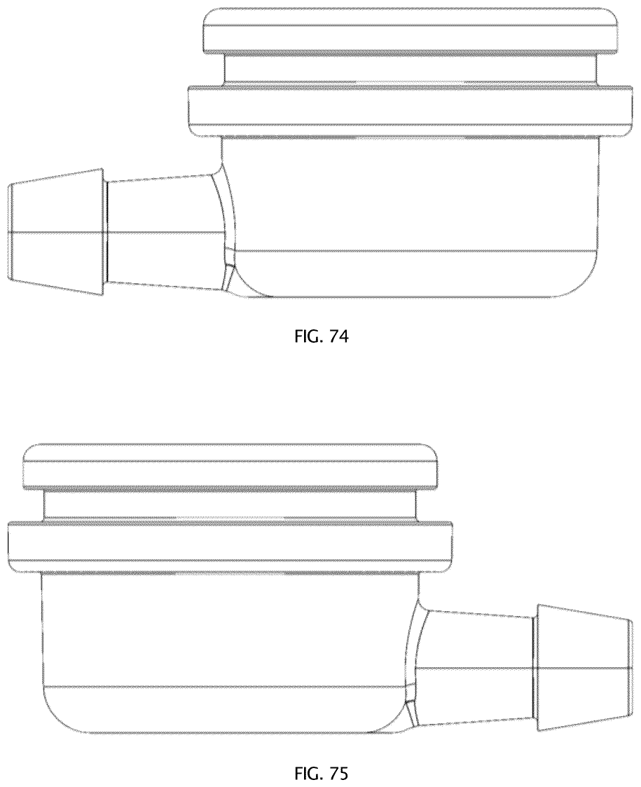

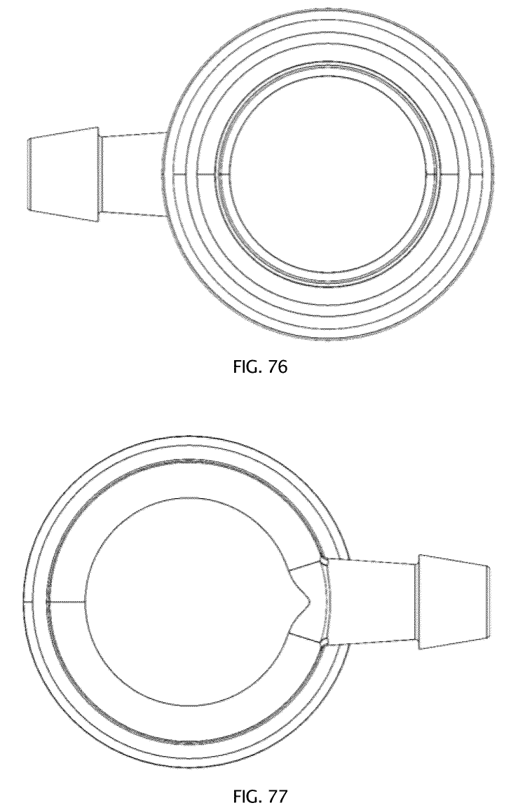

FIGS. 33-37 illustrate an alternative embodiment of a connector 800. The connector is configured to fluidly connect with an adapter 602. The adapter comprises a body 612 and a connection for fluidly connecting with a blood pressure monitor (FIGS. 2, 42). The adapter 602 comprises a cavity and a plug 604 (shown dashed in FIG. 36) in fluid communication with the connection. The plug may have a generally cylindrical shape. The adapter 602 may also comprise resilient arms 610 that include angled tabs 608.

Embodiments of the connector 800 may comprise a generally cylindrical body 802 with an internal cavity 804, an open end 806, and a closed end 808. The body may comprise a sidewall 810 having a lower end 812 adjacent a bottom wall 814 of the close end 808. The sidewall may also comprise an upper end 816. A rim 818 may extend outwardly from an outer surface 822 of the side wall 810. The rim 818 may comprise a lip 820 that extends upwardly from the rim to form a step or seat 824 adjacent to the wall tip end 816. A collar 826 may be inserted into an open end of the body and positioned into seat 824. The collar 826 may comprise an outer surface 828 an inner surface 830. A slot 832 having a lip 834 may be formed in the outer surface 828. The slot may surround the entire circumference of the outer surface 828. A slot 836 having a cavity 838 extending from an upper end of the slot may be formed in the inside surface 830 of the collar 826. A wiper or seal 840 may be positioned at least partly within the slot 836 and cavity 838. The seal 840 may comprise a lip 842 extending inwardly from the seal body 844.

When the connector 602 plug 604 is inserted into the connector collar 826, the seal lip 842 contacts an outside surface of the plug such that fluid flow is restricted or an air-tight seal is formed between the connector and the plug. In addition, as the plug is inserted, tabs 608 of the connector are pushed outward so that they allow clearance of the collar outer surface 828. Once the connector is fully seated, the resilient arms 610 return to a previous state, moving tabs 608 into the connector slot 832 where tab 608 engage with the lip 834 of the slot, securing the connector to the connector.

A barbed connector 276 may extend from the outer surface 822 of sidewall 810. The barbed connector 276 may be inserted into a lumen of a tube, for example connected to a blood pressure monitor or cuff, such that a barb 286 of the connector engages an inside surface of the lumen and secures the connector 800 to the tube. A bore 298 may extend through the barbed connector, through the connector sidewall 810, and into the cavity 804 formed within the connector. The connector cavity may have a generally cylindrical shape with a central axis 846 extending between the closed 808 and open 806 ends of the body 802. The bore 298 may have a central axis 848 extending along an axis of the barbed connector 276. The bore axis 848 and the cavity axis 846 may be positioned at angle relative to one another of between 45 and 135 degrees, and more preferably at an angle of 90 degrees.

The written description set out above describes various features and aspects of various embodiments of adapters and/or connectors for use with blood pressure monitors. However, it will be understood that the accompanying figures may illustrate additional features, elements, or functionality that, while not explicitly described, would be understood by a person having ordinary skill in the art. The figures and any features, elements, or functionality that would be understood therefrom form part of the disclosure of this application. While the present invention has been described with reference to one or more particular embodiments, those skilled in the art will recognize that many changes may be made thereto without departing from the spirit and scope of the present invention. Furthermore, components from one embodiment can be used in other non-exclusive embodiments. Each of these embodiments and obvious variations thereof is contemplated as falling within the spirit and scope of the invention.

What is claimed is:

1. A blood pressure cuff comprising:
a cuff adapted to encircle a patient's appendage, the cuff comprising a first air passageway and a second air passageway;
a tube having a first end and a second end, the tube comprising:
a first lumen connected with and in fluid communication with the first air passageway at the tube first end, the first lumen comprising a first lumen sidewall,
a second lumen connected with and in fluid communication with the second air passageway at the tube first end, the second lumen being spaced apart from the first lumen and comprising a second lumen sidewall, and
a web having a consistent length and extending from the first lumen sidewall to the second lumen sidewall such that the first lumen is spaced apart from the second lumen by a first distance, the first distance between the first lumen and the second lumen being maintained by the consistent length of the web;
a first connector attached to the first lumen at the tube second end; and
a second connector attached to the second lumen at the tube second end.

2. The blood pressure cuff of claim 1 wherein the first connector is separate and spaced apart a second distance from the second connector.

3. The blood pressure cuff of claim 2 wherein the first distance is a distance from a central axis of the first lumen to a central axis of the second lumen.

4. The blood pressure cuff of claim 3 wherein the second distance is a distance from a central axis of the first connector to a central axis of the second connector.

5. The blood pressure cuff of claim 4 wherein the central axis of the first connector is coaxial with the central axis of the first lumen, and the central axis of the second connector is coaxial with the central axis of the second lumen.

6. The blood pressure cuff of claim 4 wherein the first distance is equal to the second distance.

7. The blood pressure cuff of claim 2 wherein the second distance between the first connector and the second connector is maintained by the consistent length of the web.

8. The blood pressure cuff of claim 1 wherein the first lumen sidewall has a first thickness, and the web has a second thickness.

9. The blood pressure cuff of claim 8 wherein the first thickness is greater than the second thickness.

10. The blood pressure cuff of claim 8 wherein the second thickness is greater than the first thickness.

11. The blood pressure cuff of claim 8 wherein the first thickness is equal to the second thickness.

12. The blood pressure cuff of claim 8 wherein the second lumen sidewall has a third thickness, and the third thickness is greater than the first thickness.

13. The blood pressure cuff of claim 1 wherein the first lumen, second lumen, and web of the tube comprise a single, integrated extrusion.

14. The blood pressure cuff of claim 1 the first lumen has a first diameter, the second lumen has a second diameter, and the second diameter is greater than the first diameter.

15. A blood pressure cuff comprising:
a cuff adapted to encircle a patient's appendage, the cuff comprising a first air passageway and a second air passageway;
a tube having a first end and a second end, the tube comprising:
a first lumen connected with and in fluid communication with the first air passageway at the tube first end, the first lumen comprising a first lumen sidewall,
a second lumen connected with and in fluid communication with the second air passageway at the tube first end, the second lumen being spaced apart from the first lumen and comprising a second lumen sidewall, and
a web extending from the first lumen sidewall to the second lumen sidewall such that the first lumen is spaced apart from the second lumen by a first distance;
a first connector attached to the first lumen at the tube second end; and
a second connector attached to the second lumen at the tube second end;
wherein the first connector and second connector are configured to connect with an adapter having a first aperture and a second aperture with a portion of the first connector being inserted into the first aperture and a portion of the second connector being inserted in the second aperture.

16. The blood pressure cuff of claim 15 wherein:
the first distance is a distance from a central axis of the first lumen to a central axis of the second lumen;
the first aperture of the adapter is spaced apart from the second aperture of the adapter by a second distance;
the second distance is a distance from a central axis of the first aperture to a central axis of the second aperture; and
the first distance between the first lumen and the second lumen is maintained by the length of the web.

17. The blood pressure cuff of claim 15 wherein first connector comprises a barb that engages an inside surface of the first lumen sidewall.

18. The blood pressure cuff of claim 15 wherein first connector comprises a slot that engages a key of the adapter.

19. A blood pressure cuff comprising:
a cuff adapted to encircle a patient's appendage, the cuff comprising a first air passageway and a second air passageway;
a tube having a first end and a second end, the tube comprising:
a first lumen connected with and in fluid communication with the first air passageway at the tube first end, the first lumen comprising a first lumen sidewall,
a second lumen connected with and in fluid communication with the second air passageway at the tube first end, the second lumen being spaced apart from the first lumen and comprising a second lumen sidewall, and
a web having a consistent length and extending from the first lumen sidewall to the second lumen sidewall such that the first lumen is spaced apart from the second lumen by a first distance, the first distance between the first lumen and the second lumen being maintained by the consistent length of the web;
a first connector attached to the first lumen at the tube second end; and
a second connector attached to the second lumen at the tube second end;
wherein the first connector and second connector are configured to connect with an adapter having a first aperture and a second aperture with a portion of the first connector being inserted into the first aperture and a portion of the second connector being inserted in the second aperture.

20. The blood pressure cuff of claim 19 wherein the tube is configured the first connector and second connector are configured to be simultaneously inserted into the adapter.
